(19) United States
(12) Patent Application Publication
(10) Pub. No.: US 2002/0058321 A1
(43) Pub. Date: May 16, 2002

(54) Method of determining the activity of 1-deoxy-D-xylulose-5-phosphate reductoisomerase and 1-deoxy-D-xylulose-5-phosphate synthase

(76) Inventors: William Frank Martin, Neuss (DE); Ruediger Hain, Langenfeld (DE); Klaus-Guenther Tietjen, Langenfeld (DE); Marco Busch, Burscheid (DE); Andreas S. Kloti, Durham, NC (US)

Correspondence Address: PARADIGM GENETICS, INC, 104 ALEXANDER DRIVE, BUILDING 2, P O BOX 14528, RTP, NC, 27709-4528, US

(21) Appl. No.: 09/923,556

(22) Filed: Aug. 7, 2001

Related U.S. Application Data
(62) Division of application No. 09/449,335, filed on Nov. 24, 1999.

(30) Foreign Application Priority Data
Jul. 30, 1999 (DE) DE 199 35967.9

(51) Int. Cl. C12N 9/02; C12N 9/90; C07H 21/04; C12P 21/02; C12N 5/04

(52) U.S. Cl. 435/189; 435/233; 435/320.1; 435/410; 536/23.2; 435/69.1

(57) ABSTRACT

The invention relates to DNA which encodes Arabidopsis 1-eoxy-xylulose-5-phosphate reductoisomerase and to a method of identifying modulators of the 1-deoxy-D-xylulose-5-phosphate reductoisomerase and 1-deoxy-D-xylulose-5-phosphate synthase activity.

The invention relates to DNA which encodes for Arabidopsis 1-deoxy-D-xylulose-5 phosphate reductoisomerase, and to a method of identifying modulators of an enzyme with 1-deoxy-D-xylulose-5-phosphate reductoisomerase activity and of an enzyme with 1-deoxy-D-xylulose-5-phosphate synthase activity.

Undesired vegetation can be prevented by using herbicides. The demands made of herbicides have risen constantly with regard to activity, costs and ecofriendliness. There is therefore a need for new substances which can be developed into new potent herbicides. In general, it is normal to search for such new guide structures in greenhouse tests. However, such tests are laborious and expensive. The number of substances which can be tested in the greenhouse is, accordingly, limited.

The search is underway for plant-specific biosynthetic routes which constitute advantageous sites of action for herbicides and do not occur in animal organisms.

Recently, it has been found that plants synthesize essential plastid isoprenoids not by exploiting the mevalonate biosynthetic pathway which is found in the animal organism, but the microbial 1-deoxy-xylulose-5-phosphate biosynthetic pathway (Lichtenthaler, K. (1998), Fett/Lipid 100, 128-138; Eisenreich et al). (1998), Chemistry & Biology 5, R221-R233).

This biosynthetic pathway finally leads to the synthesis of, inter alia, carotenoids: and the side chains of plastoquinone and chlorophyll. These products are essential for the photosynthetic growth of plants, Inhibition of one step in this biosynthetic pathway entails the end of plant growth.

This is why the 1-deoxy-xylulose-5-phosphate biosynthetic pathway is of particular interest in the search for new herbicidally active compounds. In particular the two enzymes 1-deoxy-D-xylulose-5-phosphate synthase (DXPS) and 1-deoxy-D xylulose-5-phosphate reductoisomerase (DXPR) are of central importance. It has already been demonstrated that DXPS (CLA I) is essential for the development of a normal plant (Mandel et al (1996), Plant J. 9, 649-658). This discovery supports the expectation that a herbicidal compound which affects DXPS activity has a herbicidal action. Also, it has been demonstrated that bacterial DXFR is inhibited by the herbicidal compound Fosmidomycin, which is already known (Zeidler et al. (1998), Z. Naturforsch. 53, 980-986; Kuzuyama et al. (1998), Tetrahedron Lett. 39, 7913-7916). However, there are no commercially useable herbicides, which affect DXPS or DXPR activity. In the search for new, improved herbicides, both enzymes are therefore of high importance as sites of action. The 1-deoxy-xylulose-5-phosphate biosynthetic pathway also has importance in microorganisms, especially in parasitic microorganisms as, for example, bacteria or plasmodia. The treatment of infectious diseases, in particular the treatment of malaria, may be based on the inhibition of this metabolic pathway (Jomaa et al. (1999), Science 285, 1573-1576).

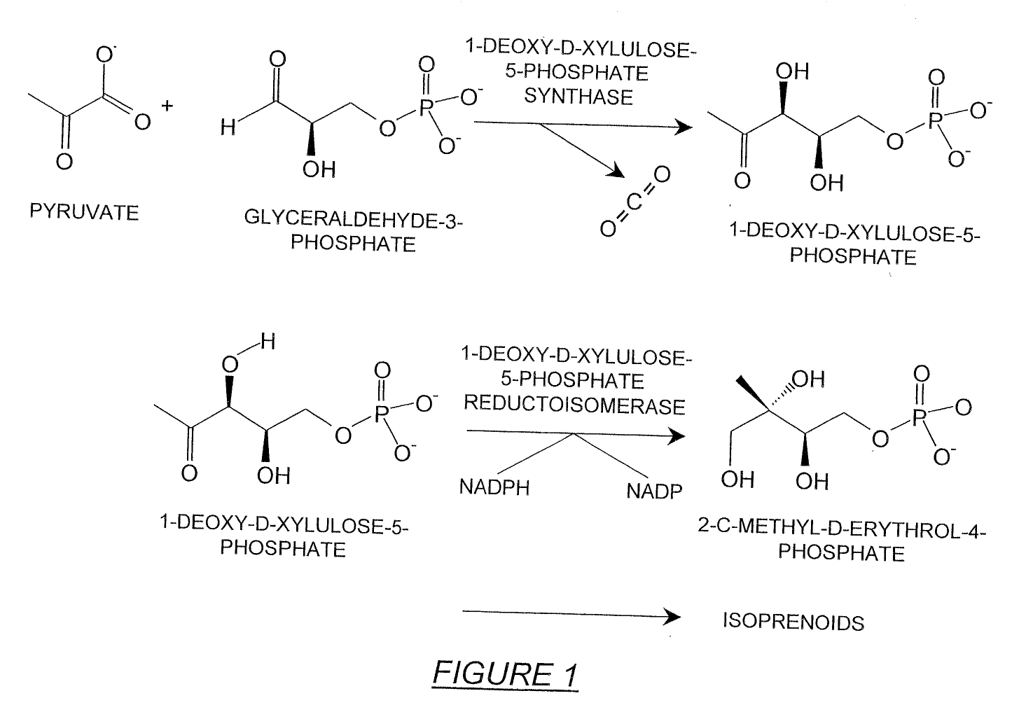

So far it has not been possible to determine the enzymatic activity of the enzyme 1-deoxy-xylulose-5-phosphate synthase in a simple test system since the reaction neither entails a measurable change in absorption nor can be coupled in a simple manner with a colour change or fluorescence change (FIG. 1). The enzymatic activity of 1-deoxy-D-xylulose-5-phosphate reductoisomerase entails a measurable change in the optical absorption of the cosubstrate NADPH. However the substrate 1-deoxy-D-xylulose-5-phosphate can only be synthesized with difficulty (FIG. 1). While 1-deoxy-D-xylulose-5-phosphate can be prepared via a chemical or biochemical route, both methods are expensive and are not well suited for use in test systems with high throughput (Taylor et al. (1998), J. Org. Chem. 63, 2375-2377; Blagg and Poulter (1999), J. Org. Chem 64,1508-1511).

The present invention solves these problems by combining the two enzymes in one test system. The Arabidopsis thaliana 1-deoxy-D-xylulose-5-phosphate synthase gene is already known under the name CLAI (Mandel et al. (1996), Plant J.5. 649-658). The 1-deoxy-D-xylulose-5-phosphate reductoisomerase gene is, as yet, known only from Mentha piperita (Lange et al. (1998), Proc. Nati. Acad. Sci. U.S.A. 5, 2100-2104) and from various microorganisms. The A. thaliana 1-deoxy-D-xylulose-5-phosphate reductoisomerase has also been described and a fragment of the amino acid sequence has been published (Lange und Croteau (1999). Archives of Biochem. and Biophys. 365, 170-174). The combined reaction of pyruvate and glyceraldehyde-3-pbosphate to give 2-C-methyl-Derythrol-4-phosphate is monitored by visually detecting the NADPH consumption (FIG. 1). The test system is suitable for the search for modulators of both enzymes, that is to say substances which inhibit or else stimulate the activity of the enzymes, and can be used for test series with high throughput (high-throughput screening, HTS). After detection of modulators by one of the two enzymes in an HTS system, the modulators of the two enzymes can be distinguished from each other by using the existing methods for measuring the activity of the two enzymes (Sprenger et al. (1997), Proc. Natl. Acad. Sci. U.S.A. 94, 12857-12862; Kuzuyama et al. (1998), Tetrahedron Lett. 39, 45094512; DE 197 52 700-A1).

The present invention relates to the DNA which encodes Arabidopsis 1-deoxy-D xylulose-5-phosphate reductoisomerase, in particular the Arabidopsis thaliana 1-deoxy-D-xylulose-5-phosphate reductoisomerase, and to fragments of this DNA, which encode functional subunits of 1-deoxy-D-xylulose-5-phosphate reductoisomerase.

The invention furthermore relates to DNA which encodes Arabidopsis 1-deoxy-D-xylulose-5-phosphate reductoisomerase, with an amino acid sequence as shown in SEQ ID NO 2 or SEQ ID NO 6.

The invention furthermore relates to DNA as described under SEQ ID NO 1 or SEQ ID NO 5 which encodes the Arabidopsis thaliana 1-deoxy-D-xylulose-5-phosphate reductoisomerase.

The invention furthermore relates to DNA which shows 80%, preferably 90%, homology to the DNA described under SEQ ID NO 1 or SEQ ID NO 5 and which encodes plant 1-deoxy-D-xylulose-5-phosphate reductoisomerase.

The invention furthermore relates to DNA which is complementary to the DNA which encodes the Arabidopsis thaliana 1-deoxy-D-xylulose-5-phosphate reductoisomerase, and to RNA which is complementary to the DNA which encodes the Arabidopsis thaliana 1-deoxy-D-xylulose-5-phosphate reductoisomerase.

The invention furthermore relates to an expression construct which encompasses DNA which encodes the Arabidopsis thaliana 1-deoxy-D-xylulose-5-phosphate reductoisomerase and is described under SEQ ID NO 1 or SEQ ID NO 5, and to a sequence which is functionally linked herewith and which allows the 1-deoxy-D-xylulose-5-phosphate reductoisomerase to be expressed.

The invention furthermore relates to a vector which comprises DNA which encodes the Arabidopsis thaliana 1-deoxy-D-xylulose-5-phosphate reductoisomerase and/or is described under SEQ ID NO 1 or SEQ ID NO 5 and which allows the 1-deoxy-D-xylulose-5-phosphate reductoisomerase to be expressed in a host cell.

The invention furthermore relates to a host cell which comprises the abovementioned DNA, an expression construct as mentioned above, or a vector which allows the 1-deoxy-D-xylulose-5-phosphate reductoisomerase to be expressed.

The invention furthermore relates to the use of DXPR and/or DXPS modulators as herbicides, antibiotic agents or anti-malarial agents.

The invention also relates to the use of DXPR and/or DXPS modulators as lead structures for the chemical optimization and the development of improved modulators.

The invention also relates to a method of determining the activity of 1-deoxy-D-xylulose-5-phosphate reductoisomerase and 1-deoxy-D-xylulose-5-phosphate synthase, which is based on combining the conversion of pyruvate and glyceraldehyde-3-phosphate to give 1-deoxy-D-xylulose-5-phosphate by 1-deoxy-D-xylulose-5-phosphate synthase with the conversion of the resulting 1-deoxy-D-xylulose-5-phosphate to give 2-C-methyl-D-erythrol4-phosphate by 1-deoxy-D-xylulose-5-phosphate reductoisomerase in one test system.

The course of the overall reaction, that is to say the conversion of the pyruvate and glyceraidehyde-3-phosphate into 2-C-methyl-D-erythrol4-phosphate is monitored with reference to the optical change by the decrease in the cofactor NADPH of 1-deoxy-D-xylulose-5-phosphate reductoisomerase.

The invention also relates to a method of identifying of substances which modify the activity of 1-deoxy-D-xylulose-5-phosphate reductoisomerase or 1-deoxy-D xylulose-5-phosphate synthase, in which the above-described test system for determining the activity of 1-deoxy-D-xylulose-5-phosphate reductoisomerase and of 1-deoxy-D-xylulose-5-phosphate synthase is used. The test system is optimized in such a way that an optimal conversion of pyruvate and glycer-aldehyde-3-phosphate to give 2-C-methyl-D-erythrol-4-phosphate is ensured. The reaction can be carried out in the presence and absence of substances which modify the activity of one of the enzymes involved. A comparison of the reaction in the presence and absence of such a substance with reference to the NADPH consumption thus allows substances which modulate, preferably inhibit, the activity of 1-deoxy-D-xylulose-5-phosphate reductoisomerase and/or of 1-deoxy-D-xylulose-5-phosphate synthase to be identified.

The invention also relates to substances which are found with the aid of the above described method, with the exception of Fosmidomycin, which is already known to inhibit 1-deoxy-D-xylulose-5-phosphate reductoisomerase (Zeidler et al. (1998), Z. Naturforsch. 53, 980-986).

The invention also relates to the use of substances which are found with the aid of the above-described method for use as modulators, preferably as inhibitors, of the enzymatic activity of 1-deoxy-D-xylulose-5-phosphate reductoisomerase and/or of 1-deoxy-D-xylulose-5-phosphate synthase.

The invention also relates to the use of substances which are found with the aid of the above-described method for use as herbicides, antibiotic agents or anti-malarial agents.

The term “functional fragments” describes those DNA fragments which encode polypeptides which still have 1-deoxy-D-xylulose-5-phosphate reductoisomerase activity, or fragments of 1-deoxy-D-xylulose-5-phosphate reductoisomerase which still have this activity.

The term “homology” in relation to DNA means that DNA segments which are at least 15 base pairs long or strands which are complementary to the DNA match the correspondingDNA in at least 80%, preferably in 90%, of the nucleotides. Such a homology is determined, inter alia, with the aid of computer programs such as the GCG program (Devereux et al. (1983), Nucleic Acids Res. 12, 387-395).

“Homology” exists also when a DNA segment is capable of hybridizing with the DNA strand in question or with its complementary strand.

The term “to hybridize” or “hybridization” describes the process in which a single stranded nucleic acid molecule undergoes base pairing with a complementary DNA strand, where the capability of a single-stranded nucleic acid molecule depends on the stringency of the hybridization conditions.

The term “stringency” relates to the hybridization conditions. “High stringency makes base pairing difficult. To do this, high temperatures of 42° C. or less are used, a formamide concentration of less than 20% and low salt (SSC) concentrations, Alternatively, temperatures of 65° C. or less can be used in combination with a low salt concentration (SSPE). “Low stringency” conditions favour the formation of base pairs. The temperatures used here are 37° C. or less, the formamide concentration is less than 50%, and the salt concentration (SSC) is moderate. Alternatively, temperatures of 50° C. or less in combination with a medium to high salt concentration (SSPE) are used.

The term “complementary” relates to the capability of purine and pyrimidine nucleotides to form base pairs with each other via hydrogen bonds. Complementary base pairs are, inter alia, guanine und cytosine, adenine and thymine, and adenine and uracil.

The term “plasmid” refers to an extrachromosomal genetic element. The original plasmids used for the present invention are either commercially available or freely accessible or can be derived from such plasmids by known methods.

The term “vector” describes a DNA element used for introducing exogenous DNA into host cells. A vector contains a nucleotide sequence which encodes one or more polypeptides.

One skilled in the art is aware of the fact that the degenerate genetic code (i.e. 64 codons encode 20 amino acids) allow a large number of “silent” substitutions of nucleotide base pairs to be introduced into the sequence shown here without changing the identity of the protein products encoded by it. The scope of the invention includes all such substitutions.

DNA Isolation

The nucleic acid mentioned here can exist in complete cells, in cell lysates, in partially purified or biologically pure form, i.e. when other cell components or chemical precursors and by-products, in the case of chemical DNA synthesis, have been removed.

The DNA mentioned here can be obtained by a series of genetic and recombinant DNA techniques, for example by means of amplification with the aid of the polymerase chain reaction (PCR) or else by de novo DNA synthesis. The DNA mentioned here can be isolated by means of PCR amplification of genomic DNA from suitable plant cells using oligonucleotide primers which are directed at a suitable region of SEQ ID NO 1 or SEQ ID NO 5 (see, for example, J. Sambrook et al, (1989), Molecular Cloning, 2nd edition, chapter 14).

Obtaining and Purifying the Protein

The invention also relates to polypeptides which have 1-deoxy-D-xylulose-5 phosphate reductoisomerase activity and which are encoded by an above-described DNA.

The skilled worker knows that the polypeptides of the present invention can be obtained by various routes, for example by chemical methods such as the solid-phase method. To obtain larger quantities of protein, the use of recombinant methods is recommended. Expression of a cloned 1-deoxy-D-xylulose-5-phosphate reductoisomerase gene or fragments thereof can take place in a series of suitable host cells which are known to the skilled worker. To this end, a 1-deoxy-D-xylulose-5 phosphate reductoisomerase gene is introduced into a host cell with the aid of known methods.

The integration of the cloned 1-deoxy-D-xylulose-5-phosphate reductoisomerase gene in the chromosome of the host cell is within the scope of the present invention. Preferably, the gene or fragments thereof are inserted into a plasmid, and the encoding regions of the 1-deoxy-D-xylulose-5-phosphate reductoisomerase gene or fragments thereof are functionally linked to a constitutive or inducible promoter.

The basic steps for generating the recombinant 1-deoxy-D-xylulose-5-phosphate reductoisomerase are:

1. Obtaining a natural, synthetic or semi-synthetic DNA which encodes 1 deoxy D-xylulose-5-phosphate reductoisomerase.

2. Introducing this DNA into an expression vector which is suitable for expressing 1-deoxy-D-xylulose-5-phosphate reductoisomerase either alone or as a fused protein.

3. Transformation of a suitable host cell, preferably a prokaryotic host cell, with this expression vector.

4. Growing this transformed host cell in a manner which is suitable for expressing 1-deoxy-D-xylulose-5-phosphate reductoisomerase.

5. Harvesting the cells and purifying 1-deoxy-D-xylulose-5-phosphate reductoisomerase by suitable known methods.

The encoding regions of 1-deoxy-D-xylulose-5-phosphate reductoisomerase and of 1-deoxy-D-xylulose-5-phosphate synthase can be expressed by the customary methods in E. coli, either separately or together. Suitable expression systems for E. coli are commercially available, for example the expression vectors of the pET series, for example pET3a, pET23a, pET28a with his-Tag or pET32a with his-Tag for the simple purification and thioredoxin fusion for improving the solubility of the expressed enzyme, and pGEX with glutathion synthetase fusion. The expression vectors are transformed into XDE34-lysogenic E. coli strains, for example, BL21(DE3), HMS 174(DE3) or AD494(DE3). After the cells have become attached, expression is induced with IPTG under standard conditions known to the skilled worker. After cell induction, incubation is carried out for 3 to 24 hours at temperatures from 18° C. to 37° C. The cells are disrupted by sonication in disruption buffer (10 to 200 mM sodium phosphate, 100 to 500 mM NaCl, pH 5 to 8). The protein which has been expressed can be purified by chromatographic methods, in the case of protein which has been expressed with a his-Tag by means of chromatography on an Ni-NTA column.

Since, according to current knowledge, the 1-deoxy-xylulose-5-phosphate pathway does not exist in animals and yeasts, expression of the protein in commercially available yeast strains (for example Pichia pastoris) or in insect cell cultures (for example Sf9 cells) is another favourable option.

Alternatively, the proteins may also be expressed in plants.

Determination of the 1-deoxy-D-xylulose-5-phosphate reductoisomerase and/or 1-deoxy-D-xylulose-5-phosphate Synthase Activity and Identification of Modulators of the Enzyme Activity of Both Enzymes

To identify and develop herbicidally active substances, it is necessary to find a way of determining the effect of various candidate substances on 1-deoxy-D-xylulose-5 phosphate reductoisomerase and 1-deoxy-D-xylulose-5-phosphate synthase activity. To this end, a way must be found of measuring the activity of both enzymes in an efficient and simple manner, or of detecting an inhbition or else stimulation of this activity.

One way of determining the effect of a substance on the enzymatic reaction of 1-deoxy-D-xylulose-5-phosphate reductoisomerase and/or of 1-deoxy-D-xylulose-5 phosphate synthase is to contact purified 1-deoxy-D-xylulose-5-phosphate reductoisomerase and 1-deoxy-D-xylulose-5-phosphate synthase, or fragments with 1-deoxy-D-xylulose-5-phosphate reductoisomerase activity and fragments with 1-deoxy-D-xylulose-5-phosphate synthase activity, with a test substance and to check the activity of both enzymes.

According to the present invention, the 1-deoxy-D-xylulose-5-phosphate reductoisomerase and 1-deoxy-D-xylulose-5-phosphate synthase activity is determined in a combined test system which contains both 1-deoxy-D-xylulose-5 phosphate reductoisomerase and 1-deoxy-D-xylulose-5-phosphate synthase. In this test system, 1-deoxy-D-xylulose-5-phosphate synthase converts pyruvate and glyceraidehyde-3-phosphate into 1-deoxy-D-xylulose-5-phosphate, which is converted by 1-deoxy-D-xylulose-5-phosphate reductoisomerase directly into 2-C -methyl-D-erythrol4-phosphate, with consumption of NADPH. The decrease in the NADPH concentration can be monitored with aid of optical measurement methods (FIG. 1).

To test for substances which modulate, preferably inhibit, the 1-deoxy-D-xylulose-5-phosphate reductoisomerase and/or 1-deoxy-D-xylulose-5-phosphate synthase activity, the test system and the enzyme concentrations are designed in such a way that an optimal conversion of the pyruvate and glyceraldehyde-3-phosphate to give 2-C-methyl-D-Erythrol-4-phosphate is ensured. If one of the enzymes involved is inhibited or activated by a candidate substance, this can be detected by a drop or increase in the NADPH conversion.

Then, a separate activity test for 1-deoxy-D-xylulose-5-phosphate reductoisomerase or 1-deoxy-D-xylulose-5-phosphate synthase may be carried out in the known manner in order to determine which of the two enzymes is affected in its activity by the substance which has been found.

As an alternative to the known, separate activity tests, HPLC makes it possible to determine, starting from the combined activity test, which products or intermediates starting from the materials pyruvate and glyceraldehyde-3-phosphate have been formed. If 1-deoxy-D-xylulose-5-phosphate has been formed, but not 2-C-methyl-D erythrol-4-phosphate, then it was DXPR which was inhibited. If 1-deoxy-D-xylulose-5-phosphate has not been formed either, the DXPS was (also) inhibited

### EXAMPLE 1

Construction of a Vector for Expressing the Arabidopsis thaliana 1-deoxy-D-xylulose-5-phosphate Synthase

The encoding sequence of the Arabidopsis thaliana CLA1 gene from position +1 to +2154 was amplified with the aid of the PCR technique using the following primers of the sequences shown in SEQ ID NO. 3 and SEQ ID NO. 4 (Mandel et al. (1996) CLAI, a novel gene required for chloroplast development, is highly conserved in evolution; Plant J. 5 649-658; Lange M. et al. (1998) A family of transketolases that directs isoprenoid biosynthesis via a mevalonate-independant pathway; Proc. Natl. Acad. Sci, U.S. A. 95, 2100-2104).

The template used was Arabidopsis thaliana single-stranded cDNA from 4-week-old seedlings.

The amplified fragment which carries the encoding sequence of the Arabidopsis thaliana DXPS was then cleaved with the restriction enzymes BamHI and NotI. The resulting BamHI/NotI-DXPS fragment was ligated into the linearized and dephosphorylated bacterial expression vector pET32 a (+) (Novagen). The resulting construct pET32-DXPS contains the encoding DXPS sequence within the reading frame with a fragment of the bacterial thioredoxin gene.

### EXAMPLE 2

Construction of a Vector for Expressing the Arabidopsis thaliana 1-deoxy-D-xylulose-5-phosphate Reductoisomerase

The encoding sequence of the Arabidopsis thaliana 1-deoxy-D-xylulose-5-phosphate reductoisomerase was amplified with the aid of the PCR technique using the primers with the sequences as shown in SEQ ID NO. 3 and SEQ ID NO. 4. The amplified fragment was then cleaved with the restriction endonucleases EcoRI and SalI. The resulting EcoRI/SalI-DXR fragment was ligated into the linearized and dephosphorylated bacterial expression vector pET32 a (+) (Novagen). In the resulting construct pET32-DXR the encoding DXR sequence was within the reading frame together with a fragment of the bacterial thioredoxin gene.

### EXAMPLE 3

Test System for Identifying 1-deoxy-D-xylulose-5-phosphate Reductoisomerase and/or 1-deoxy-D-xylulose-5-phosphate Synthase Modulators in an HTS

A microtitre plate (96-well format) is filled with solutions of 1-deoxy-xylulose-5 phosphate synthase (0. 1-10 μg of purified enzyme/100 μl) and 1-deoxy-xylulose-5 phosphate reductoisomerase (0.1-10 μg of purified enzyrne/100 μl) and of the cosubstrate NADPH (0.1-10 mM) in the customary buffer (10-200 MM sodium phosphate pH 5-8), containing thiamine diphosphate (0.1-10 nM) and MgCl₂ (0.5-50 mM). All concentrations (also those given further below) are based on the concentration after addition of all assay components. The candidate chemical, or as control, buffer, is pipetted into each cavity of the microtitre plate. After addition of the substrates pyruvate (1-100 mM) and glyceraldehyde-3-phosphate (1-100 mM) in customary buffer (as above, but without NADPH and without thiamine diphosphate), the plates are incubated between 18° C. and 45° C. until a drop in the optical density of NADPH at 340 nm which can be measured easily has been reached. The optical density is then read in a customary microtitre plate reader. Substances which inhibit one of the two enzymes which participate are identified by a reduced drop of the NADPH concentration with the aid of the optical density measurement.

In microtitre plates of greater density (384-well, 1536-well format and the like), the volumes indicated above are adapted to suit the system

Explanation of the Figure and of the Sequence Data

FIG. 1 shows the conversion of pyruvate and glyceraldehyde-3-phosphate via 1-deoxy-D-xylulose-5-phosphate to give 2-C-methyl-D-erythrol4-phosphate, which is catalysed by the enzymes 1-deoxy-D-xylulose-5-phosphate synthase and 1-deoxy D-xylulose-5-phosphate reductoisomerase. The reaction which is catalysed by 1-deoxy-D-xylulose-5-phosphate reductoisomerase requires NADPH as cofactor. The catalysed reactions are essential for the isoprenoid synthesis in plants.

SEQ ID NO. 1

DNA sequence encoding the Arabidopsis thaliana 1-deoxy-D-xylulose-5-phosphate reductoisomerase. The other sequence shown is the amino acid sequence encoded by the DNA.

SEO ID NO. 2

Amino acid sequence of the Arabidopsis thaliana 1-deoxy-D-xylulose-5-phosphate reductoisomerase with 477 amino acids.

SEQ ID NO. 3

Oligonucleotide derived from the encoding sequence of the Arabidopsis thaliana CLAI gene for amplifying the Arabidopsis thaliana CLAI gene by means of the PCR technique, including nucleotides for a BamHI cloning site.

SEQ ID NO. 4

Oligonucleotide derived from the encoding sequence of the Arabidopsis thaliana CLAI gene for amplifying the Arabidopsis thaliana CLAI gene by means of the PCR technique, including nucleotides for a NotI cloning site.

SEO ID NO. 5

Another DNA sequence encoding the Arabidopsis thaliana 1-deoxy-D-xylulose-5-phosphate reductoisomerase. Change at nucleotides 890-892. The other sequence shown is the amino acid sequence encoded by the DNA.

SEQ ID NO. 6

Another amino acid sequence of the Arabidopsis thaliana 1-deoxy-D-xylulose-5-phosphate reductoisomerase with 477 amino acids. Change at amino acid 292.

SEO ID NO. 7

Oligonucleotide derived from the encoding sequence of the Arabidopsis thaliana 1-deoxy-D-xylulose-5-phosphate reductoisomerase gene for amplifying the Arabidopsis thaliana 1-deoxy-D-xylulose-5-phosphate reductoisomerase gene by means of the PCR technique.

SEQ ID NO. 8

Oligonucleotide derived from the encoding sequence of the Arabidopsis thaliana 1-deoxy-D-xylulose-5-phosphate reductoisomerase gene for amplifying the Arabidopsis thaliana 1-deoxy-D-xylulose-5-phosphate reductoisomerase gene by means of the PCR technique.

1. DNA which encodes Arabidopsis 1-deoxy-D-xylulose-5-phosphate reductoisomerase or functional fragments thereof.

2. DNA according to claim 1, characterized in that the DNA encodes the total, or functional fragments of an amino acid sequence as shown in SEQ ID NO. 2.or SEQ ID NO. 6.

3. DNA according to claim 1 or 2, characterized in that the DNA encodes Arabidopsis thaliana 1-deoxy-D-xyl ulose-5-phosphate reductoisomerase.

4. DNA according to any of claims 1 to 3 with a sequence as shown in SEQ ID NO. 1, SEQ ID NO. 5 or fragments thereof.

5. DNA, characterized in that its homology to the sequence as shown in SEQ ID NO. 1 or SEQ ID NO. 5 is at least 80%.

6. DNA according to claim 5, characterized in that its homology to the sequence as shown in SEQ ID NO. 1 or SEQ ID NO. 5 is at least 90%.

7. DNA, characterized in that it is complementary to the DNA according to any of claims 1 to 6.

8. RNA, characterized in that it is complementary to the DNA according to any of claims 1 to 6.

9. Expression construct, characterized in that it encompasses DNA as claimed in any of claims 1 to 6 and a sequence which is functionally linked herewith and which allows the DNA to be expressed.

10. Vector, characterized in that it contains DNA according to any of claims 1 to 7 and 9.

11. Host cell containing DNA according to any of claims 1 to 7, an expression construct according to claim 9 or a vector according to claim 10.

12. Use of 1-deoxy-D-xylulose-5-phosphate reductoisomerase and/or 1-deoxy-D-xylulose-5-phosphate synthase modulators as herbicides, antibiotic agents or anti-malarial agents.

13. Method of determining the activity of 1-deoxy-D-xylulose-5-phosphate reducloisomerase and/or 1-deoxy-D-xylulose-5-phosphate synthase, characterized in that
(a) pyruvate and glyceraldehyde-3-phosphate are converted into 1-deoxy-D-xylulose-5-phosphate with the aid of 1-deoxy-D-xylulose-5-phosphate synthase, and
(b) the resulting 1-deoxy-D-xylulose-5-phosphate is converted into 2-C-methyl-D-erythrol4-phosphate with the aid of 1-deoxy-D-xylulose-5-phosphate reductoisomerase.

14. Method according to claim 13, characterized in that the conversion of 1-deoxy-D-xylulose-5-phosphate into 2-C-methyl-D-erythrol-4-phosphate is measured with reference of the NADPH consumed during this conversion.

15. Method according to claim 13 or 14, characterized in that the 1-deoxy-D xylulose-5-phosphate reductoisomerase activity is determined.

16. Method according to claim 13 or 14, characterized in that the 1-deoxy-D xylulose-5-phosphate synthase activity is determined.

17. Method of identifying substances which modify the activity of 1-deoxy-D xylulose-5-phosphate reductoisomerase and/or 1-deoxy-D-xylulose-5 phosphate synthase, characterized in that the method according to any of claims 13 to 16 is carried out in the presence and absence of a test substance, and the activities found in each case are compared with each other.

18. Substances which are identified by a method according to claim 17, with the exception of Fosmidomycin.

19. Use of substances which are identified by a method according to claim 17 as modulators of the enzymatic activity of 1-deoxy-D-xylulose-5-phosphate reductoisomerase and/or of 1-deoxy-D-xylulose-5-phosphate synthase.

20. The use of substances identified by a method according to claim 18 as herbicides, antibiotic agents or anti-malarial agents.

21. Polypeptide represented by SEQ ID NO. 2 or SEQ ID NO. 6.


SEQUENCE LISTING (8 sequences)

SEQ ID NO: 1 - DNA, 1732 bp, Arabidopsis thaliana
  CDS (18)..(1448): DNA encoding 1-deoxy-D-xylulose-5-phosphate reductoisomerase
ttttttaaaagactctgatgatgacattaaactcactatctccagctgaatccaaagctatttctttcttggatacctccaggttcaatccaatccctaaactctcaggtgggtttagtttgaggaggaggaatcaagggagaggttttggaaaaggtgttaagtgttcagtgaaagtgcagcagcaacaacaacctcctccagcatggcctgggagagctgtccctgaggcgcctcgtcaatcttgggatggaccaaaacccatctctatcgttggatctactggttctattggcactcagacattggatattgtggctgagaatcctgacaaattcagagttgtggctctagctgctggttcgaatgttactctacttgctgatcaggtaaggagatttaagcctgcattggttgctgttagaaacgagtcactgattaatgagcttaaagaggctttagctgatttggactataaactcgagattattccaggagagcaaggagtgattgaggttgcccgacatcctgaagctgtaaccgttgttaccggaatagtaggttgtgcgggactaaagcctacggttgctgcaattgaagcaggaaaggacattgctcttgcaaacaaagagacattaatcgcaggtggtcctttcgtgcttccgcttgccaacaaacataatgtaaagattcttccggcagattcagaacattctgccatatttcagtgtattcaaggtttgcctgaaggcgctctgcgcaagataatcttgactgcatctggtggagcttttagggattggcctgtcgaaaagctaaaggaagttaaagtagcggatgcgttgaagcatccaaactggaacatgggaaagaaaatcactgtggactcctgtacgcttttcaacaagggtcttgaggtcattgaagcgcattatttgtttggagctgagtatgacgatatagagattgtcattcatccgcaaagtatcatacattccatgattgaaacacaggattcatctgtgcttgctcaattgggttggcctgatatgcgtttaccgattctctacaccatgtcatggcccgatagagttccttgttctgaagtaacttggccaagacttgacctttgcaaactcggttcattgactttcaagaaaccagacaatgtgaaatacccatccatggatcttgcttatgctgctggacgagctggaggcacaatgactggagttctcagcgccgccaatgagaaagctgttgaaatgttcattgatgaaaagataagctatttggatatcttcaaggttgtggaattaacatgcgataaacatcgaaacgagttggtaacatcaccgtctcttgaagagattgttcactatgacttgtgggcacgtgaatatgccgcgaatgtgcagctttcttctggtgctaggccagttcatgcatgaagaattggttgttggaagaacataaggaagcttctgaggaaatgttgaaagaagattagtgtagagaatggggtactacttaatagcgtttttggcaaggattatggattgtgtagctaatttatctgtgatccgaacaagccaaactgataatttgaaaccattgtgatccgaacaagccaaactgataatttgaaaccatttttaccaataaaaccgagcttaattgtttcacattatatgattaattacattcatctaagggttcttgaaaaaaaaaa

SEQ ID NO: 2 - protein, 477 aa, Arabidopsis thaliana
MMTLNSLSPAESKAISFLDTSRFNPIPKLSGGFSLRRRNQGRGFGKGVKCSVKVQQQQQPPPAWPGRAVPEAPRQSWDGPKPISIVGSTGSIGTQTLDIVAENPDKFRVVALAAGSNVTLLADQVRRFKPALVAVRNESLINELKEALADLDYKLEIIPGEQGVIEVARHPEAVTVVTGIVGCAGLKPTVAAIEAGKDIALANKETLIAGGPFVLPLANKHNVKILPADSEHSAIFQCIQGLPEGALRKIILTASGGAFRDWPVEKLKEVKVADALKHPNWNMGKKITVDSCTLFNKGLEVIEAHYLFGAEYDDIEIVIHPQSIIHSMIETQDSSVLAQLGWPDMRLPILYTMSWPDRVPCSEVTWPRLDLCKLGSLTFKKPDNVKYPSMDLAYAAGRAGGTMTGVLSAANEKAVEMFIDEKISYLDIFKVVELTCDKHRNELVTSPSLEEIVHYDLWAREYAANVQLSSGARPVHA

SEQ ID NO: 3 - DNA, 30 bp, Arabidopsis thaliana
cctaggatccatggcttcttctgcatttgc

SEQ ID NO: 4 - DNA, 29 bp, Arabidopsis thaliana
atatgcggccgctcaaaacagagcttccc

SEQ ID NO: 5 - DNA, 1732 bp, Arabidopsis thaliana
  CDS (18)..(1448): new coding sequence
ttttttaaaagactctgatgatgacattaaactcactatctccagctgaatccaaagctatttctttcttggatacctccaggttcaatccaatccctaaactctcaggtgggtttagtttgaggaggaggaatcaagggagaggttttggaaaaggtgttaagtgttcagtgaaagtgcagcagcaacaacaacctcctccagcatggcctgggagagctgtccctgaggcgcctcgtcaatcttgggatggaccaaaacccatctctatcgttggatctactggttctattggcactcagacattggatattgtggctgagaatcctgacaaattcagagttgtggctctagctgctggttcgaatgttactctacttgctgatcaggtaaggagatttaagcctgcattggttgctgttagaaacgagtcactgattaatgagcttaaagaggctttagctgatttggactataaactcgagattattccaggagagcaaggagtgattgaggttgcccgacatcctgaagctgtaaccgttgttaccggaatagtaggttgtgcgggactaaagcctacggttgctgcaattgaagcaggaaaggacattgctcttgcaaacaaagagacattaatcgcaggtggtcctttcgtgcttccgcttgccaacaaacataatgtaaagattcttccggcagattcagaacattctgccatatttcagtgtattcaaggtttgcctgaaggcgctctgcgcaagataatcttgactgcatctggtggagcttttagggattggcctgtcgaaaagctaaaggaagttaaagtagcggatgcgttgaagcatccaaactggaacatgggaaagaaaatcactgtggactctgctacgcttttcaacaagggtcttgaggtcattgaagcgcattatttgtttggagctgagtatgacgatatagagattgtcattcatccgcaaagtatcatacattccatgattgaaacacaggattcatctgtgcttgctcaattgggttggcctgatatgcgtttaccgattctctacaccatgtcatggcccgatagagttccttgttctgaagtaacttggccaagacttgacctttgcaaactcggttcattgactttcaagaaaccagacaatgtgaaatacccatccatggatcttgcttatgctgctggacgagctggaggcacaatgactggagttctcagcgccgccaatgagaaagctgttgaaatgttcattgatgaaaagataagctatttggatatcttcaaggttgtggaattaacatgcgataaacatcgaaacgagttggtaacatcaccgtctcttgaagagattgttcactatgacttgtgggcacgtgaatatgccgcgaatgtgcagctttcttctggtgctaggccagttcatgcatgaagaattggttgttggaagaacataaggaagcttctgaggaaatgttgaaagaagattagtgtagagaatggggtactacttaatagcgtttttggcaaggattatggattgtgtagctaatttatctgtgatccgaacaagccaaactgataatttgaaaccattgtgatccgaacaagccaaactgataatttgaaaccatttttaccaataaaaccgagcttaattgtttcacattatatgattaattacattcatctaagggttcttgaaaaaaaaaa

SEQ ID NO: 6 - protein, 477 aa, Arabidopsis thaliana
MMTLNSLSPAESKAISFLDTSRFNPIPKLSGGFSLRRRNQGRGFGKGVKCSVKVQQQQQPPPAWPGRAVPEAPRQSWDGPKPISIVGSTGSIGTQTLDIVAENPDKFRVVALAAGSNVTLLADQVRRFKPALVAVRNESLINELKEALADLDYKLEIIPGEQGVIEVARHPEAVTVVTGIVGCAGLKPTVAAIEAGKDIALANKETLIAGGPFVLPLANKHNVKILPADSEHSAIFQCIQGLPEGALRKIILTASGGAFRDWPVEKLKEVKVADALKHPNWNMGKKITVDSATLFNKGLEVIEAHYLFGAEYDDIEIVIHPQSIIHSMIETQDSSVLAQLGWPDMRLPILYTMSWPDRVPCSEVTWPRLDLCKLGSLTFKKPDNVKYPSMDLAYAAGRAGGTMTGVLSAANEKAVEMFIDEKISYLDIFKVVELTCDKHRNELVTSPSLEEIVHYDLWAREYAANVQLSSGARPVHA

SEQ ID NO: 7 - DNA, 32 bp, Arabidopsis thaliana
cctagaattcatgacattaaactcactatctc

SEQ ID NO: 8 - DNA, 30 bp, Arabidopsis thaliana
atatgtcgactcatgcatgaactggcctag